(19) United States
(12) Patent Application Publication
(10) Pub. No.: US 2009/0054881 A1
(43) Pub. Date: Feb. 26, 2009

(54) MAMMALIAN BIOFILM TREATMENT PROCESSES AND INSTRUMENTS

(76) Inventor: Yosef Krespi, New York, NY (US)

Correspondence Address: IP Patent Docketing;K&L GATES LLP, 599 Lexington Avenue, 33rd Floor, New York, NY, 10022-6030, US

(21) Appl. No.: 12/139,295

(22) Filed: Jun. 13, 2008

Related U.S. Application Data
(60) Provisional application No. 60/944,007, filed on Jun. 14, 2007.
(60) Provisional application No. 61/023,595, filed on Jan. 25, 2008.

(51) Int. Cl. A61B 18/20; A61C 17/00

(52) U.S. Cl. 606/9; 606/14; 433/29; 433/216

(57) ABSTRACT

A process for treatment of biofilm resident or present at a mammalian treatment site applies shockwaves to remove, disrupt, disperse, dislodge, destroy or attenuate the biofilm. The shockwaves can be generated in a handheld instrument by impinging a laser on a suitable target material. Removal of biofilm from implantable surgical devices is also described.

### CROSS-REFERENCE TO A RELATED APPLICATION

This application claims the benefit of provisional application No. 60/944,007 filed Jun. 14, 2007 and of provisional application No. 61/023,595 filed Jan. 25, 2008. The disclosure of each one of said provisional applications Nos. 60/944,007 and 61/023,595 is incorporated by referenced herein.

### STATEMENT REGARDING FEDERALLY SPONSORED RESEARCH OR DEVELOPMENT

(Not applicable.)

The present invention relates to processes and instruments for treating biofilms resident in mammals and includes processes and instruments for treatment of undesired mammalian biofilms to control the biofilms.

### BACKGROUND

Biofilms are ubiquitous and can be problematic. Some examples of common biofilms include dental plaque, drain-clogging slime and the slippery coating found on rocks in streams and rivers.

Industrial and commercial problems attributable to biofilms include corrosion of pipes, reduced heat transfer and/or reduced hydraulic pressure in industrial cooling systems, the plugging of water injection jets and the clogging of water filters. In addition, biofilms can cause significant medical problems, for example, by infecting host tissues, by harboring bacteria that contaminate drinking water, and by causing rejection of medical implants.

Biofilms are generally formed when bacteria and/or other microorganisms adhere to surfaces in aqueous environments and begin to excrete a slimy, adhesive substance that can anchor the microorganisms to a wide variety of materials including metals, plastics, soil particles, medical implant materials and animal tissue.

A biofilm is often a complex aggregation of microorganisms comprising a protective and adhesive matrix generated by excretion of polymeric materials, for example, polysaccharides, from the microorganisms. Biofilms are often attached to surfaces, have structural heterogeneity and genetic diversity, and exhibit complex community interactions. Their protective matrix and genetic diversity mean that biofilms are often hard to destroy or otherwise control and conventional methods of killing bacteria, such as antibiotics, and disinfectants, are often ineffective against biofilms.

Because the single cell microorganisms in a biofilm typically are in an attached state, closely packed together and secured to each other and to a solid surface, they are more difficult to destroy than when they are in a free-floating mobile mode, as is the case in many mammalian infections.

A number of proposals have been made for the chemical or pharmaceutical treatment of, or regulation of, the growth of mammalian-resident biofilms. However, as implied above, such methods may be ineffective or subject to resistance or both, or may have other drawbacks commonly associated with pharmaceuticals such as systemic action and side effects.

Some suggestions for treatment of biofilms in humans appear in the patent literature. For example, Bornstein U.S. Patent Application Publication No. 2004/0224288 (referenced “Bornstein” herein) discloses a system and process for thermolytic eradication of bacteria and biofilm in the root canal of a human tooth employing an optical probe and a laser oscillator.

Also, Hazan et al. U.S. Patent Application Publication No. 2005/0261612 discloses a method for decreasing materials such as biofilm attached to a mammalian body which method includes attaching a nanovibrational energy resonator device onto an external or internal area of the body.

Oxley et al. “Effect of ototopical medications on tympanostomy tube biofilms.” Laryngoscope. 2007 October; 117(10):1819-24 describes experiments to examine the effect of ototopical medications on biofilms on fluoroplastic tympanostomy tubes. Reportedly, microbial activity in colony forming units (CFU) was decreased after three weeks. However, despite the treatment, the biofilm was not eradicated but continued to grow. The authors conclude that infectivity of the biofilm can be temporarily neutralized by antibiotic ototopicals and that the biofilm may progress despite treatment.

International patent publication No. WO 00/67917 describes a method for permeabilizing biofilms using stress waves to create transient increases in the permeability of the biofilm. As described, the increased permeability facilitates delivery of compounds, such as antimicrobial or therapeutic agents into and through the biofilm, which agents are apparently to be employed to treat the biofilm.

Desrosiers et al. “Methods for removing bacterial biofilms: in vitro study using clinical chronic rhinosinusitis specimens.” Am J Rhinol. 2007 September-October; 21(5):527-32 describes an in vitro study on removed biofilms from bacterial isolates obtained from patients with refractory chronic rhinosinusitis. As described, the biofilm was treated with both static and pressurized irrigation and a citric acid/zwitterionic surfactant. According to the authors, the pressurized treatment employing irrigant and a surfactant can disrupt the biofilms tested.

Notwithstanding the foregoing proposals, it would be desirable to have new processes and treatments for treatment of biofilms resident in or on mammalian sites.

The foregoing description of background art may include insights, discoveries, understandings or disclosures, or associations together of disclosures, that were not known to the relevant art prior to the present invention but which were provided by the invention. Some such contributions of the invention may have been specifically pointed out herein, whereas other such contributions of the invention will be apparent from their context. Merely because a document may have been cited here, no admission is made that the field of the document, which may be quite different from that of the invention, is analogous to the field or fields of the present invention. Nor is any admission made that the document was published prior to, or otherwise predates, applicant's invention.

### SUMMARY OF THE INVENTION

In one aspect, the present invention provides a process for treatment of an undesired biofilm resident at a treatment site in or on a mammalian host. The process can comprise applying shockwaves to the biofilm resident at the treatment site to control the biofilm. Pursuant to the invention, control of the biofilm can comprise reducing the mass of, removing, disrupting, attenuating or destroying the biofilm. For example, the biofilm can be ablated or disintegrated, or eliminated.

In one embodiment of the invention, control of the biofilm comprises applying the shockwaves to the biofilm to cause one or more pieces of the biofilm to tear away from the residual biofilm or from the treatment site. In another embodiment, control of the biofilm comprises oscillating the biofilm and oscillating it may lead to pieces breaking away.

Generally, the biofilm comprises material and/or organisms foreign to the mammalian host, and the invention comprises controlling such material and/or organisms foreign to the mammalian host rather than controlling host tissue by disintegration or the like. Some embodiments of the invention control the application of shockwaves to maintain host tissue at the treatment site intact or free of visible or otherwise apparent symptoms of heat or other damage, or both intact and free of symptoms of heat damage. Application of shockwaves to biofilm at a treatment site can be effected with delivery of little if any heat to host tissue or other host structure.

Mammalian biofilms are often, or usually, undesired, and can sometimes lead to medical complications if not treated effectively. Accordingly, useful embodiments of the invention provide a simple and effective treatment process, and a treatment instrument for performing the process, that can be applied to control internal or external mammalian treatment sites where biofilms are present. Internal treatment sites can be accessed via bodily cavities, for example the nostrils, or subcutaneously, employing a catheter, trocar or the like, or in other ways.

Shockwaves or pressure pulses to be applied to the treated biofilm can be generated using light energy, for example, light energy output by a laser, or by other suitable means, or the shockwaves can be generated in another suitable manner. In one embodiment of the invention, the shockwaves are applied by impinging a laser beam on to an ionizable, optionally metallic, target to generate mechanical shockwaves. Optionally, the process can include pulsing the laser beam. A plasma can be formed adjacent the ionizable target the mechanical shockwaves emanating from the plasma can be generated.

While the invention is not limited by or dependent upon any particular theory, it appears from such experiments that the shockwaves employed in some embodiments of the invention may be sufficiently powerful to break up a biofilm, and possibly dislodge it from its support structure, without causing visible damage to the underlying tissue, implant or other host structure. For example, in vitro experiments described herein show that a biofilm can be removed from a suture fiber, without visibly apparent structural damage to the delicate filaments of the suture fiber.

In one embodiment of the invention, to avoid tissue heating injury, which may manifest itself in only a few seconds of heat exposure, the process can employ a laser-induced shockwave treatment instrument which propagates little or no heat externally of the instrument. Also, or alternatively, a shockwave treatment instrument can be employed which propagates little or no laser energy externally of the instrument.

The process can also comprise irrigating the treatment instrument, the treatment site, or both, to remove detritus from the treatment instrument and/or the treatment site, if desired. An aqueous fluid can be employed for irrigation. Optionally, the aqueous fluid can be pulsed.

Biofilms that can be treated by a process according to the invention may be resident or on or at any of a variety of anatomical sites and include biofilms secured to the treatment site by polysaccharide material. The biofilms can comprise one or more microorganisms species selected from the group consisting of bacteria, fungi, protozoa, archaea and algae.

In vitro experiments described herein show that a biofilm grown on an implantable surgical device can be caused to oscillate and break up, and can possibly be destroyed, employing laser-induced shockwaves as can be utilized in the practice of the invention. In some cases a biofilm can be more or less completely removed from its site of residence. Another in vitro experiment described herein shows a shockwaves treatment in accordance with the invention causing a substantial killing of bacteria with a colony count reduction of about 50 percent. Embodiments of the inventive processes and instruments can be applied in a variety of fields including, for example, for cleaning biofilm-contaminated cardiac implants and associated devices and materials.

The invention includes mammalian host implants cleaned of biofilm by a treatment process according to the invention.

In another aspect, the invention provides a treatment instrument for effecting photodestruction of or controlling an undesired biofilm resident at a treatment site in or on a mammalian host. The treatment instrument can be employed to apply shockwaves to the treatment site to destroy the biofilm. One embodiment of the treatment instrument comprises an ionizable target for transducing laser energy into shockwaves and an optical fiber extending along the treatment instrument and having a distal end positioned adjacent the ionizable target. The optical fiber can be connectable with a pulsed laser energy source to receive pulses of laser energy from the laser energy source and discharge the pulses of laser energy from the distal end of the optical fiber to impinge on the ionizable target, outputting shockwaves.

### BRIEF DESCRIPTION OF THE SEVERAL VIEWS OF THE DRAWING

Some embodiments of the invention, and of making and using the invention, as well as the best mode contemplated of carrying out the invention, are described in detail herein and, by way of example, with reference to the accompanying drawings, in which like reference characters designate like elements throughout the several views, and in which:

FIG. 1 is a schematic view of laser generation of shockwaves from the distal tip of a treatment instrument useful in the practice of the invention;

FIG. 2 is a graph showing schematically the effects of various laser treatments that are generally obtainable at different power densities, energy densities and application times;

FIG. 3 is an image of a culture plate to which a biofilm is attached;

FIG. 4 is an image of the culture plate shown in FIG. 7 during disruption of the biofilm by a shockwave treatment according to an embodiment of the invention;

FIG. 5 is a composite image comprising a view of a stainless steel orthopedic screw (center), an enlarged view of a portion (indicated by the large arrow) of the stainless steel screw to which a biofilm is attached (view A on the left) and a similarly enlarged view of the portion of the stainless steel screw during disruption of the biofilm by a shockwave treatment according to an embodiment of the invention (view B on the right);

FIG. 6 is an enlarged image of the portion of the stainless steel screw shown in FIG. 9, from a different angle, with attached biofilm before shockwave treatment;

FIG. 7 is an enlarged image of the portion of the stainless steel screw shown in FIG. 9 during shockwave treatment;

FIG. 8 is an enlarged image of the portion of the stainless steel screw shown in FIG. 9 after shockwave treatment;

FIG. 9 is a side view of a suture fiber;

FIG. 10 is an enlarged end view image of the suture fiber shown in FIG. 9 with a biofilm attached, a 600 μm scale being shown;

FIG. 11 is an image similar to FIG. 14 of the suture fiber after disruption of the biofilm by a shockwave treatment according to an embodiment of the invention;

FIG. 12 is an image of a tympanostomy tube with a biofilm attached;

FIG. 13 is an image similar to FIG. 16 of the tympanostomy tube during disruption of the biofilm by a shockwave treatment according to an embodiment of the invention;

FIG. 14 is an image similar to FIG. 17 of the tympanostomy tube after further shockwave treatment; and

FIG. 15 is an enlarged image of the tympanostomy tube shown in FIGS. 16-18 after disruption of the biofilm by the shockwave treatment.

### DETAILED DESCRIPTION OF THE INVENTION

Biofilms can form in mammalian hosts when bacteria adhere to a wet surface and begin to excrete a slimy, glue-like substance that can anchor the bacteria to tissue or medical implants. Such biofilms can comprise many types of bacteria, fungi, debris and corrosion products. Biofilms encountered in the human or other mammalian body generally comprise matter which is foreign to the mammalian host. Generally, biofilms do not comprise host tissue and are not useful components of the mammalian host. Thus, embodiments of the invention may apply treatments to host tissue on which biofilm resides or which are in the vicinity of biofilms but generally do not aim to change or modify the host tissue or other host structure subject to treatment. One embodiment of the invention comprises controlling or attenuating biofilm foreign matter while leaving host tissue intact. Useful embodiments of the invention target biofilms which may actively or passively adversely affect normal functioning of the mammalian host.

Non-living surfaces in the body, for example catheters, contact lenses, artificial joints and other medical devices may be more prone to biofilm formation than living tissue. However, biofilms can also grow on living tissue, and may cause diseases such as endocarditis, lung, dental, sinus, ear and other infections. For example, it is believed that biofilms may play an etiologic role in chronic otolaryngologic infections. Therapeutic methods designed to treat acute infections caused by surface or floating (planktonic) microorganisms may be found to be ineffective for chronic infections when biofilms are present.

Bacteria can adhere to solid surfaces and excrete a slimy, slippery coat with structured features. The resulting adherent mass can be referred to as a bacterial biofilm. The formation of biofilm structure occurs in multiple stages. First the bacteria may attach to a convenient, usually wet, surface. The attachment may be strengthened by a polymeric matrix adhering densely to the surface, and an aggregation of micro colonies occurs. The environment can provide growth and maturation for the biofilm which becomes an organized structure. Finally, during its mature phases, the biofilm may detach, disperse or embolize to perform the same cycle in adjacent or distant areas.

The composition of a biofilm can comprise, for example, about 15% by weight of bacteria cells and about 85% by weight of ‘slime’. The slimy environment also appears to protect the bacteria from natural host defenses such as inflammatory cells, antibodies and antimicrobial treatments. As the biofilm cells consume nutrients from surrounding tissue and fluids, nutrient gradients develop until bacteria near the center or centers of the biofilm become starved and go into quiescent state. It is speculated that this dormancy may partially explain the resistance often displayed by biofilm bacteria to antibiotics which are effective against rapidly growing bacteria in standard tests. The biofilm bacteria survive in a matrix rich in extracellular polymeric substances (“EPS” herein) including polysaccharides, nucleic acids and proteins providing a protective and nutritious environment to the microorganisms.

Some examples of virulent bacteria that may be found in biofilms treatable by the processes and instruments of the invention, with diseases with which they are associated indicated in parenthesis, are: Pseudomonas aeruginosa (cystic fibrosis); Staphylococcus aureus (osteomyelitis); Proteus vulgaris (pyelonephritis); Streptococcus viridans (endocarditis); culture-negative prostatitis; and Haemophilus influenzae (otitis media).

It is also believed that a biofilm can have a complex morphology comprising communication channels in which cells in different regions of the biofilm exhibit different patterns of gene expression. It may have a three dimensional architecture with open channels that allow the transport of nutrients into the biofilm. Furthermore, bacteria in biofilms may communicate through quorum sensing molecules that can coordinate and up-regulate virulence factors when cells became starved. Quorum sensing, or exchange of molecules, genes, DNA and free communication between cells, can provide the bacteria within the biofilm a resistant and protective environment. Known anti-bacterial agents may require a hundred- or thousand-fold ‘normal’ antibiotic dosage to be effective against such resistant biofilm structures; which is not feasible to administer systemically owing to toxicity.

Biofilms can provide a mechanism for microorganisms to survive extreme temperature changes, radiation or mechanical trauma. Antibiotics may eradicate planktonic (floating or drifting) microorganisms, and possibly also surface bacteria on a biofilm without damaging bacteria protected within the polymer matrix. This understanding may point to a role of biofilms in the etiology of chronic infections with acute exacerbations. Some examples in otolaryngology include chronic rhinosinusitis, chronic otitis media, adenoiditis and cryptic tonsillitis. A given condition may be aggravated by the presence of a prosthetic, implantable device or catheter for example a tympanostomy tube, a tracheotomy tube, a cochlear implant, a stent, packing material or a foreign body. Biofilms preferentially form in grooves, depressions, pockets and other surface discontinuities on host-resident medical devices and implants. Biofilms can also form between or on the fibers of sutures, on cuffs and in the mesh-like structures of knitted or woven grafts. The literature reports having found a dense biofilm in the surface depressions of a cochlear implant removed from a patient with an intractable infection. These and other sites where biofilms are attached, resident or supported can constitute treatment sites to be subjected to shockwave treatments in embodiments of the processes of the present invention.

Not all biofilms are pathogenic. However even non-pathogenic biofilms can create an inflammatory reaction in surrounding host tissue and may cause collateral damage through cytotoxic, proteolytic, and proinflammatory effects. These effects may cause localized tissue reactions and recurrent infections. Sometimes, the host response to a biofilm can result in severe and sustained inflammation. For example, in diseases such as cystic fibrosis and gingivitis, if the neutrophils fail to engulf the bacteria inside biofilms, they may degranulate and damage host tissues.

The processes of the invention described herein usefully can be employed in the treatment of biofilms resident in mammals, including in particular, humans. In addition, these processes can be applied to treatment of non-human mammals including, for example, horses, cattle, sheep, llamas, husbanded animals, pets including dogs and cats, laboratory animals, for example, mice, rats and primates, animals employed for sports, breeding, entertainment, law enforcement, draft usage, zoological or other purposes, if desired. The processes and devices of the invention are not limited by the theories of biofilm formation and structure described herein or by any other theories.

Processes according to the invention can be employed to treat biofilms resident at, adhered to, or otherwise present at any of a variety of anatomical sites, including any one or more sites selected from the group consisting of otolaryngological sites; nasal, sinus, and middle ear cavities; pharyngal, tonsillar, dental and periodontal sites; toenails and fingernails and their environment; sites on cardiac implants, endovascular implants, orthopedic implants, gynecological implants, intrauterine devices, urologic implants, urinary catheters, therapeutic and other implants as will be or become apparent to a person of ordinary skill in the art. The invention provides treatment instruments adapted to treat a biofilm present at any one or more of the foregoing sites by a process according to the invention.

The biofilm treatment processes of the invention can provide complete or partial elimination of, attrition of, removal or reduction of, photodestruction of or other desired control of, or biofilm resident in or on a host mammal, in particular, a human being. Processes according to the invention can treat undesired biofilms which may cause the host to be symptomatic and in some cases can lead to medical complications.

As summarized above the invention provides biofilm treatment processes which comprise applying shockwaves to a biofilm resident at a treatment site on or in a mammalian host.

In one embodiment of the invention, the shockwaves generated are non-convergent shockwaves and the process can comprise directing the non-convergent shockwaves on to the biofilm resident at the treatment site.

Processes according to the invention can employ a treatment instrument to generate the shockwaves, and the treatment instrument can have an ionizable target and a distal tip. The treatment instrument can impinge a laser beam on to the target to generate shockwaves which can be mechanical in nature and can comprise disturbances in a fluid medium. The distal tip can comprise a metallic target and the plasma can be formed at the distal tip. One embodiment of the process, or method, comprises inserting the distal tip of the treatment instrument into the mammalian body to be treated and effecting the application of shockwaves to a biofilm while the distal tip is inserted into the mammalian body.

Processes according to the invention can comprise manipulating the treatment instrument, optionally by hand, to direct the shockwaves on to the biofilm resident at the treatment site. For example, such a process can comprise translating the treatment instrument across the biofilm to incrementally destroy the biofilm. If desired, the treatment instrument can be translated across the biofilm in multiple passes.

In some embodiments of the invention the treatment device comprises an inspection fiber and the process includes inserting the inspection fiber into the mammalian body. An operator can then monitor the treatment by viewing the treatment site via the inspection fiber and manipulating the treatment instrument according to what is viewed.

If desired, the distal tip of the treatment instrument can be inserted into a bodily cavity or introduced subcutaneously. For this purpose, a flexible treatment instrument and a catheter, or trocar or the like, can be employed and the process can comprise inserting the treatment instrument into the vascular system using the catheter or trocar or other suitable device.

In another embodiment of the invention, the treatment instrument can comprise a distal port and the method can comprise outputting the shockwaves through the distal port. The process can comprise manipulating the treatment instrument to position the distal port to be at a distance in the range of from about 0.5 mm to about 10 mm. from the biofilm at the treatment site and applying the shockwaves to the biofilm with the treatment instrument so spaced from the biofilm.

Some embodiments of process according to the invention can comprise controlling the biofilm non-thermolytically or by avoiding delivery of heat to the treatment site or without applying stain to the biofilm or according to a combination of two or all of the foregoing parameters. In other embodiments, the process can comprise controlling the application of shockwaves to maintain host tissue at the treatment site intact or free of symptoms of heat or other damage or both intact and free of symptoms of heat damage.

The process can comprise employing a treatment instrument to apply the shockwaves and employing aspiration to locate the treatment instrument relatively to the biofilm at the treatment site. In a further embodiment of the invention the process can employ aspiration or suction to locate the treatment instrument relatively to the treatment site. Suction can also be applied to the treatment site to aspirate dislodged debris and irrigant from the treatment site, whether or not it is employed to locate the treatment instrument relatively to the treatment site.

Another embodiment of process according to the invention comprises controlling the application of shockwaves to the biofilm by selection of one or more control parameters selected from the group consisting of laser energy pulse width, pulse repetition rate, pulse energy and total energy delivered to the target site, the distance of the output port from the target site and the fiber-to-target distance.

A further embodiment of process according to the invention comprises pulsing the laser energy impinged on the target to have a pulse width in the range of from about 2 ns to about 20 ns, a pulse rate of from about 0.5 Hz to about 200 Hz, a pulse energy in a range of from about 2 mJ to about 15 mJ of energy per pulse and a fiber-to-target distance in the range of from about 0.7 to about 1.5 mm.

In some cases a single treatment can be effective to provide adequate photodestruction, disruption or dispersal of the biofilm. Multiple passes may be employed in the course of a single treatment. In some embodiments of the invention an individual treatment wherein shockwaves are being applied to a biofilm is performed in less than five minutes and the interval during which shockwaves are applied to the biofilm can be no more than two minutes or, possibly, one minute. During this interval, a desired number of shockwave pulses is targeted at the biofilm, which number can be in the range of from about 5 to about 100 pulses, for example in the range of from about 10 to about 50 pulses. In some cases such a single treatment can more or less completely disrupt, disperse or destroy the biofilm.

The invention also includes processes wherein a biofilm infection or infestation is treated repeatedly at intervals, for example, of from about four hours to about a month. The treatments can, if desired be repeated at intervals of from about 1 to about 14 days. Treatments can be repeated until adequate control of the biofilm, and of recurrence of the biofilm, are obtained, if desired. A course of treatment can, for example, endure for from about two weeks to about twelve months or for another suitable period.

The term “shockwave” as used herein is intended to include unsteady pressure fluctuations or waves having a speed greater than the speed of sound. Also included are pressure waves having a speed greater than the speed of sound which comprise a disturbed region in which abrupt changes occur in the pressure, density, and velocity of the medium through which the pressure wave is traveling.

The processes of the invention can employ any suitable treatment instrument which can apply shockwaves, pressure pulses or other suitable non-chemical mechanical or energetic forces to mammalian biofilms to destroy them partially or completely, without unacceptable damage to host tissue, for example, so that the tissue at the treatment site remains intact. The energetic forces can be generated by laser or other photic means, piezoelectrically or in another desired manner.

Some examples of treatment instruments suitable for the practice of the present invention include surgical instruments such as are disclosed in Dodick et al. U.S. Pat. Nos. 5,906,611 and 5,324,282 (referenced as “the Dodick instrument” herein). The disclosure of each of the Dodick et al. patents is incorporated by reference herein. Some uses and modifications of the Dodick instrument which also can be useful in the practice of the present invention are disclosed in Thyzel U.S. Patent Application Publication No. 2007/0043340 (referenced as “Thyzel” herein). The disclosure of Thyzel is also incorporated by reference herein.

As described by Dodick et al., the Dodick instrument is a laser-powered surgical instrument that employs a target for transducing laser energy into shockwaves. The instrument can be used in eye surgery, particularly for cataract removal which is effected by tissue fracturing. The Dodick instrument can comprise a handpiece holding a surgical needle and an optical fiber extending through a passageway in the needle. An open distal aspiration port for holding tissue to be treated communicates with the passageway through the needle. An optical fiber can extend along the length of the needle and have its distal end positioned close to a metal target supported by the instrument. Also as described by Dodick et al., pulses of laser energy are discharged from the distal end of the optical fiber to strike the target. The target, which can be formed of titanium metal, is described as acting as a transducer converting the electromagnetic energy to shockwaves that can be directed onto tissue in an operating zone adjacent to the aspiration port. If desired, the needle can be flexible to enhance access to treatment sites.

As described in the literature, such laser generated shockwave technology can be used in cataract surgery for extraction and photolysis of the lens and for the prevention of secondary cataract formation. The technology can be used in surgical methods which gently break-up the cloudy lens into tiny pieces that can be removed through an aperture of the probe. Using several hundred pulses, resulting in high pressures the object can be cracked efficiently with low energy deposition and without significant temperature changes around the needle.

According to M. Iberler et al. “Physical Investigations of the A.R.C.-Dodick-Laser-Photolysis and the Phacoemulsification”, unlike ultrasonic energy cataract treatments, this type of instrument produces no clinically significant heat at the incision site, when employed for cataract surgery. Apparently, the heat created within the tip of the instrument can be dissipated by heat transport in the solid titanium target.

Some embodiments of the present invention can employ the shockwaves generated at the instrument's distal port, to impinge on and destroy, attenuate, disrupt or dislodge a host-resident biofilm attached to host tissue, to an implant surface or to another treatment surface located in the operating zone adjacent the treatment instrument's distal port. The process can be performed with or without aspiration through the treatment instrument's distal port or through another port in the treatment instrument or another device.

The shockwaves output can be directed at a biofilm or other target, and in some embodiments of the invention can be applied in an identifiable approximate pattern such as a circle, an ellipse or a comparable shape, or a portion of such a pattern. The shockwaves can be output as a non-convergent shockwave beam confined to be directional. For example the shockwave beam can be divergent and can have a generally conical or other suitable shape. The divergence of the shockwave beam, defined by opposed outer edges of the beam can be from about 0° to about 900 for example from about 5° to about 30°. Such a non-convergent shockwave beam can be useful for controlled application of shockwaves on selected areas of a treatment site.

While the invention is not limited by any particular theory, it is believed that the application of mechanical shockwaves or other pressure pulses will burst the cell walls of at least some of the organisms in the treated biofilm, destroying the organisms. Unlike chemical or pharmaceutical processes which may have little effect on dormant organisms that may have very low metabolic rates, the shockwaves employed are expected, in some cases, also to destroy such dormant organisms that receive the full effect of a shockwave output from the treatment instrument. Destruction of organisms that are actually or potentially resistant to antibiotics is contemplated to be achievable, in some cases. Accordingly, in some cases where the biofilm infection is readily accessible, substantial elimination of the biofilm can be feasible. Multiple treatments can be useful to obtain a desired attrition of a particular biofilm.

Also, the treatment processes of the invention can be controlled to be non-damaging to host tissue or to cause only modest, acceptable damage compatible with the seriousness of the infection. This is unlike the process described by Dodick et al. which comprises fracturing the tissue.

Similarly, it is contemplated that the inventive treatment processes can be performed with little, if any, pain being inflicted on the host mammal. In the case of severe or persistent biofilm infections, higher intensity shockwave dosages, which can cause minor discomfort or modest pain, may be acceptable.

At sensitive treatment sites, or in other situations where more gentle treatments are desired, less frequent repetition rates or pressure pulses below shockwave intensity can be employed. For gentle treatments, single pulses at desired intervals, or pulse repetition rates in the range of from about 1 to about 10 Hz, or other desired patterns of repetition, or mild conditions, can be employed, if desired.

In some embodiments of the inventive treatment process, the distal port of the treatment instrument from which shockwaves or other mechanical pulses are output can be translated across the biofilm during the treatment process. Such translation can be effected by linear movement of the treatment instrument relatively to the biofilm, by relative rotational movement, or by combinations of the two. Varying the rate of translation or the pattern of translation, or both, provides a surgeon or other operator a useful parameter for controlling the intensity of application. For example, the treatment instrument can be reciprocated back and forth, with or without rotational movements in juxtaposition to the target biofilm and can output shockwaves in a directional beam so that the directional shockwave beam sweeps back and forth across the target biofilm, ablating the target biofilm progressively with each sweep. If desired, the requisite manipulations can be visually guided according to observation of depletion of the biofilm employing a visual aid such as is described herein.

Other parameters the operator can adjust to help manage a treatment are described elsewhere herein or will be or become apparent to a person of ordinary skill in the art in light of this disclosure. Where helpful to protect local tissue, the biofilm can, if desired, be treated in multiple passes whereby incremental attrition or destruction of the biofilm is achieved.

As described in the Dodick et al. patents, the passageway in the needle of the Dodick instrument can be used for infusion of saline or for aspiration of saline and tissue. In practicing the present invention, this passageway can be employed for irrigation of the treatment site with saline or other suitable fluid or for aspiration of the fluid and debris, including biofilm remnants produced by application of mechanical shockwaves to the biofilm at the treatment site. In general, it is not anticipated that tissue fragments will be present or aspirated, although in some cases they may be.

In various embodiment of the treatment processes of the invention, the passageway in the treatment instrument can be employed for aspiration and a separate instrument can be employed for irrigation. In other embodiments of the treatment process of the invention, the passageway in the treatment instrument can be employed for irrigation and a separate instrument can be employed for aspiration. In further embodiments of the treatment processes of the invention, the treatment instrument is provided with passageways for both irrigation and aspiration.

A process embodiment of the invention comprises slow downstream irrigation of the fiber tip to keep it clean and to remove detritus without the use of suction.

The laser energy pulses employed to induce the shockwaves or pressure pulses used in the biofilm treatment processes of the invention can be provided by any suitable laser. For example, as described by Dodick et al., a neodymium-doped yttrium-aluminum-garnet laser (“neodymium-YAG” or “ND:YAG”) laser providing light energy at a wavelength of 1,064 nanometers with a pulse width of approximately 8 nanoseconds (“ns” herein) and an absorption coefficient in water of 0.014/mm can be employed. Alternatively, other laser types can be employed, for example, gas lasers or solid lasers.

The laser energy pulses can be provided with any suitable characteristics including pulse width, pulse repetition rate and pulse energy. A pulse width or pulse duration in the range of from about 2 ns to about 20 ns can be employed, for example from about 4 ns to about 12 ns. A pulse rate of from about 0.5 Hz to about 50 Hz, for example from about 1 Hz to about 10 Hz can be employed. Higher pulse rates up to about 100 or 200 pulses per second can be employed, if desired. Any suitable pulse energy can be employed, for example, in a range of from about 2 to about 15 millijoules (“mJ”) of energy per pulse. Some embodiments of the invention can employ a pulse duration of from about 8 to about 12 nanoseconds, a repetition rate of from about 2 to about 6 pulses per second and/or an energy per pulse of from about 6 to about 12 millijoules.

In some cases, utilizing such parameters, from about 200 to about 800 shockwave-generating laser energy pulses can be employed to effectively treat a biofilm or a portion of a biofilm addressed by the distal port of the treatment instrument, without significant tissue or other damage. However, depending upon the area of biofilm to be treated, more or less laser energy pulses may be effective, for example from 5 pulses to 1500 pulses can be employed. For example, smaller treatment sites such as the ethmoid sinus can be effectively treated with a smaller number of pulses, for example less than 200 pulses. Comparably, larger treatment sites, for example a maxillary sinus can be treated with a greater number of pulses, for example 500 or more pulses, and if the area of the site so indicates, more than 800 pulses.

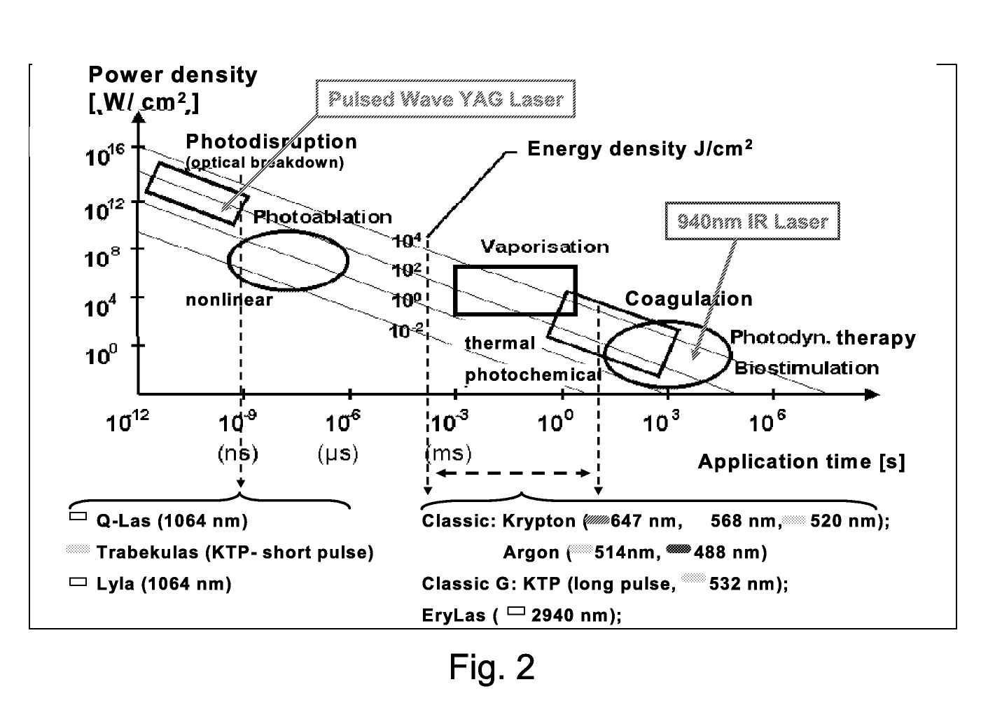

While, as noted herein, the invention is not limited by any particular theory, FIG. 2 helps explain how a pulsed YAG laser, or comparable laser or other energy source, can be employed in embodiments of the present invention to generate high intensity shockwaves of short duration that can be employed to control a biofilm resident in a mammalian host without significant damage to tissue or other host structure supporting or in the vicinity of the biofilm.

FIG. 2 provides a graphic indication of the comparative effects of a number of different therapeutic treatments comprising the application of laser or laser-generated energy to tissue. In general, the therapeutic effect of a particular energy treatment of mammalian tissue and of possible collateral damage will be functions of the nature and quantity of energy delivered and the distribution of the energy over space and time. An excessive concentration of energy in space and time may result in tissue damage, for example, from undue heating.

In FIG. 2, laser energy application time in seconds and power density in watts/cm² are plotted on the “X” and “Y” scales respectively while energy density in J/cm² is plotted on a diagonal scale. All the scales employed are logarithmic so that small graphic differences on each scale may correspond with substantial quantitative differences in the energy parameters depicted. A number of different laser energy technologies is referenced beneath the “X” scale and their approximate time scales are indicated.

As may be seen from FIG. 2, in general, classical laser technologies such as visible wavelength krypton, argon and long pulse KTP (potassium titanyl phosphate) lasers, as well as longer and shorter infrared lasers, employ relatively low power densities and long application times. These technologies can have useful applications such as for vaporization, coagulation, photodynamic therapy and biostimulation.

More recently developed lasers such as Q-switched lasers and short-pulse KTP lasers and the like employ relatively higher power densities and shorter application times. These technologies can have useful applications such as for photoablation and photodestruction. As shown by an arrow in the upper lefthand corner of FIG. 2, a pulsed YAG laser outputting in the infrared, such as can be employed in practicing the present invention, employs a notably high power density, for example, in excess of 1012 watts/cm², and a notably short application time, for example measured in nanoseconds or less. Because the higher power density may be applied for a quite short time, the energy density with such a use of a pulsed YAG laser can be comparable with that of classical lasers, namely around 10² joule/cm², give or take an order of magnitude. The energy density may also depend upon the particular geometry of the application.

The Dodick instrument can be modified as appropriate for use in any one or more process embodiments of the present invention. If desired, the invention can include a treatment instrument or a range or kit of treatment instruments adapted for treatment of particular treatment sites. For example, the distal end of the treatment instrument can be elongated to be received into a subject's nostril for treatment of the upper nasal cavity or can be further elongated for treatment of one or more sinus cavities. For treatment of one or more sinus cavities, the distal end of the treatment instrument can be sufficiently thin and elongated to be received into the nose and access a desired sinus cavity.

For treatment of cardiac, orthopedic, gynecologic, urologic or other implants, the treatment instrument can be adapted for catheter delivery of the distal tip of the treatment instrument to a treatment site via a suitable blood vessel or vessels, for example, an artery. Alternatively, the treatment instrument can be appropriately modified for subcutaneous delivery, for example, for laparoscopic delivery. The invention includes biofilm treatment processes wherein the treatment instrument is delivered via a catheter, or laparoscopically, or in other suitable manner.

In some embodiments of the invention, the treatment instrument can comprise an inspection fiber to view the treatment site and monitor the progress of the treatment. This capability can be useful for treatment sites which are unexposed or concealed including internal sites such as the upper nose and sinuses and implant surfaces. The inspection fiber can have a distal input end disposable in the vicinity of the applicator needle tip to survey the treatment site and a proximal output end communicating optically with an output device viewable by a surgeon or other operator performing the treatment. The output device can be a video screen, an optic member, or another viewing element. If desired, the inspection fiber can extend through or alongside the treatment instrument or can comprise a separate device. Also if desired, the treatment instrument with the inspection fiber can be inserted into a bodily cavity or through an incision to access a treatment site. The inspection fiber can enable the operator to monitor the treatment and manipulate the treatment instrument accordingly.

In one embodiment of the invention the tip of the treatment instrument along with an optical fiber can be incorporated into a flexible endoscope suitable for subcutaneous catheter delivery and optical imaging can be employed to enable treated sites to be visually monitored.

In some embodiments of the processes of the present invention, one or more of a number of treatment parameters to facilitate or improve performance of the treatment can be adjusted and improved or optimized for a particular application, for example by manipulation of an appropriate control, or instrument or other device by the surgeon or other operator. These parameters include the orientation, location and/or disposition of the treatment instrument, the application of saline or other irrigation fluid, the application of suction, and any one or more of the energy parameters employed to generate the applied pressure pulses. The energy parameters include the intensity, frequency, and pulse duration of the pressure pulses.

In the treatment of concealed treatment sites, adjustment of the treatment parameters can be facilitated by providing illumination means at the treatment site to illuminate the treatment site, as described herein. This measure can permit the surgeon, or other operator, to adjust one or more of the treatment parameters according to what he or she sees at the treatment site. Accordingly, some embodiments of the invention comprise illuminating the treatment site.

One embodiment of treatment instrument useful for practicing the invention is illustrated in the drawings. Other embodiments will be, or become, apparent to a person of ordinary skill in the art in light of the disclosure herein.

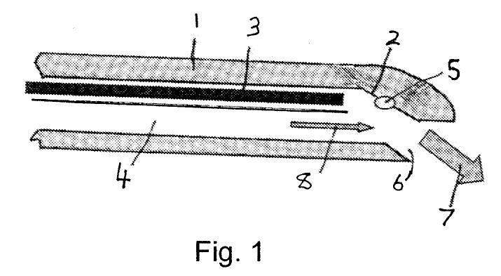

Referring to FIG. 1 of the drawings, the distal tip 1 of the treatment instrument comprises a titanium or stainless steel target 2, an optical fiber 3 which terminates adjacent target 2 and a passage 4 for irrigation fluid. Pulsed laser energy propagated along optical fiber 3 strikes target 2 causing ionization of the target material and inducing a plasma 5. Laser-induced plasma 5 causes a shockwave to be generated and to exit the treatment instrument through opening 6 in the direction of the arrow 7. Irrigation fluid supplied in the direction of arrow 8 can clean and remove debris from target 2 and the treatment site.

Titanium is useful as a target material for the purposes of the invention, for its good bio-compatibility and high absorption coefficient with respect to the laser wavelength and for its thermal conductivity. The latter properties can be useful in avoiding propagation of laser energy or heat externally of the treatment instrument, which could adversely impact sensitive tissue at the treatment site. Other embodiments of the invention can employ stainless steel, zirconium or another suitable target material.

In one embodiment of the treatment instrument shown in FIG. 1 opening 6 has a diameter of about 0.8 mm, distal tip 1 has a width of about 1.4 mm and the distance between the end of optical fiber 3 and target 2, the fiber-to-target distance, is in the range of from about 0.7 to about 1.5 mm, for example about 1 mm.

Any suitable laser system can be employed to provide laser energy to optical fiber 3 of the treatment instrument illustrated in FIG. 1. One example of a suitable laser system comprises a Nd:YAG laser operating in the infrared at a wavelength of 1064 nm, which can be Q-switched to provide high intensity energy pulses, if desired. Using an optical fiber 3 of diameter 283 Mm, the Nd:YAG laser system can be employed to generate pulsed laser energy with a pulse length of about 4 ns (nanoseconds), a frequency of from about 1 to about 10 Hz and with an energy of from about 10 to about 15 mJ. If desired, the laser system can include a control computer and a video display to monitor performance.

One treatment process utilizing the illustrated treatment instrument comprises inspecting a treatment site harboring a biofilm or otherwise diagnosing a condition appropriate for treatment by a laser-induced shockwave process according to the invention and determining a suitable treatment protocol. For example, distal tip 1 of the illustrated treatment instrument is then inserted into the bodily cavity constituted by the patient's nostril, through the naris, and is manipulated to address the internal bodily site to be treated, for example a sinus.

When the illustrated treatment instrument is properly positioned, the laser source is activated to supply a desired dosage of laser pulses along optical fiber 3. In one embodiment of the invention the treatment site is positioned in front of opening 6 of the treatment instrument. The laser energy strikes target 2 of distal tip 1, generating a shockwave in the direction of arrow 7 which is applied to the treatment site. The shockwave can be generated in the ambient fluid medium, air, irrigation fluid or the like, on the same side of target 2 as is impinged by the laser beam. In some cases, the shockwave has a direction of propagation which is approximately in the direction of reflection of the laser beam from the target surface or, rather, is in the direction the laser beam would have been reflected if not absorbed by the target.

Desirably, during treatment, the distance from the closest point of distal tip 1 to the treatment site is in the range of from about 0.5 mm to about 10 mm, for example from about 1 mm to about 5 mm, and so far as is practical, the distance is maintained, for example by suitable manipulation of the treatment instrument by the user.

It is contemplated that the effect of the laser-induced shockwaves impacting on biofilm present at the target site, including biofilm adhered to tissue at the treatment site, will be to attenuate, disrupt, disperse or weaken the biofilm or to cause the biofilm to lose its integrity or lose adherence to its substrate or to cause one or more pieces to break away. Multiple ones of these results may occur and the biofilm may be destroyed partially or entirely. Dosages can be increased and treatments can be repeated to increase biofilm attrition, if desired. Dosages can be controlled to limit collateral tissue damage or inflammation which it is believed can be controlled to be little or modest, or not visibly apparent, employing dosages such as are described herein.

Subsequently to, or concurrently with, application of laser-induced shockwaves, irrigation fluid can be supplied via an irrigation connector (not shown) and irrigation passage 4 to remove debris including biofilm detritus, if generated, and clean distal tip 1 and the treatment site.

If desired, an endoscope (not shown) can be employed with the illustrated treatment instrument to view the treatment process and treatment site and the endoscope may comprise a video camera or other suitable optics. The endoscope can be used simultaneously with the use of the illustrated treatment instrument to apply shockwaves or it can be employed to inspect the treatment site before and after treatment. Also, if desired, the illustrated treatment instrument can be modified for endoscopic delivery to the treatment site.

If desired, the laser-induced shockwave treatments of the invention can be accompanied by or followed by local or systemic administration of an antibiotic to limit or control possible infection associated with dispersal of the targeted biofilm. Treatment apparatus according to the invention can include a treatment instrument such as the illustrated treatment instrument and a laser system selected and tuned to supply appropriate laser energy to the illustrated treatment instrument. The treatment apparatus can also include associated computing and display equipment and, optionally, an endoscope for treatment site inspection, process monitoring and/or instrument delivery.

### Example of Biofilm Destruction In Vitro

Biofilms are grown from a clinical otorhea isolate of Pseudomonas aeruginosa PittDYFP. PittDYFP is a construct which constitutively expresses yellow fluorescent protein and has gentamicin as a selective marker. The biofilms are grown for 72 hours in 1/10th strength Luria-Bertani (LB) broth (Difco) with 25 μg/ml gentamicin in the presence of four different potential substrates. The potential substrates comprise MATTEK (trademark) glass bottomed culture plates (MatTek Corporation, Ashland, Mass.) configured with a glass-to-plastic step and three types of implantable surgical device, namely 316L stainless steel (316LSS) orthopedic screws (Synthes), a fluoroplastic tympanostomy tube (or ear ventilation tube) and polyethylene terephthalate (“PET” herein) suture fibers are placed in 20 ml Falcon tubes. The medium is replaced daily and the growth period is 3 days. The cultures are incubated at 37° C. in a humidified 5% CO₂ atmosphere on a shaker table at 100 RPM.

To prepare the cultures for imaging, the medium is replaced with sterile Ringer's solution to remove loosely attached and planktonic cells. The surgical devices are aseptically removed from the Falcon tubes, placed in 35 mm diameter Petri plates and immersed in Ringer's solution. The biofilms generated are imaged before, during and after laser shockwave application using a Leica TCS SP2 AOBS confocal upright DMRXE7 microscope with either a 10× air objective or a long working distance 63×0.90 n.a. water immersion lens. The biofilm is imaged either growing in the grooves of the screw threads, around the tympanostomy tube, inside and between the filaments of the PET suture fibers or on the glass-to-plastic step of the culture plates. In some cases the biofilms are stained with propidium iodide (Molecular Probes) according to the manufacturer's instructions or at 1/10th recommended strength.

The sample cultures are treated with laser-generated shockwaves employing a pulsed Nd:YAG laser at a wavelength of 1064 nm. The laser output energy is between about 8 mJ and about 12 mJ. The laser is pulsed using passive Q-switch pulsing with a pulse length between about 4 ns and about 8 ns. The laser energy is delivered to the biofilms using a handpiece intended for cataract surgery such as is described in Dodick U.S. Pat. No. 5,906,611. As described in the Dodick patent, in the handpiece, an optical fiber tip outputting laser pulses is aimed at a titanium target producing plasma and generating a shockwave.

Distally, the handpiece employed comprises a disposable needle or probe instrument in the form of a hollow metal 1.2 mm diameter tube coupled with an optical fiber of diameter about 300 μm at one end and with a 0.7 mm opening at the other end. The laser beam propagates axially inside the tube and hits a titanium target, positioned adjacent and above the opening at the tip of the probe to output shockwaves through the opening. The handpiece has a passageway for irrigation fluid which outputs adjacent the shockwave opening.

To apply shockwaves to the samples, the handpiece is moved toward the samples and then maintained at a distance of about 5 mm to 10 mm from the biofilm while operating the laser to generate shockwaves. The shockwaves are initiated by a series of low energy laser pulses in a slow stream of irrigation liquid. A 488 nm laser is used to excite the yellow fluorescent protein and 488 nm and 543 nm laser lines are used to excite the propidium iodide treated samples.

The stainless steel surfaces of the orthopedic screws and the glass-to-plastic surfaces of the culture plates, as well as the tympanostomy tube and the PET suture fibers are imaged using reflected light from the 488 nm laser line. The biofilms are also imaged with transmitted light. Before and after images are taken in the same locations using surface features such as the screw grooves or scratches as fiducial points.

During treatment with the Nd:YAG laser a time-lapse imaging function is used to capture images in the transmitted mode. Image rendering is effected by confocal stacks and time series are rendered using Imaris BITPLANE (trademark) image rendering software.

During exposure to the shockwaves generated by the Nd:YAG laser each biofilm can be seen to oscillate in response to laser pulses directed at the biofilm from a distance in excess of about 10 mm. As the handpiece approaches the target area to a distance of about 5 mm to about 10 mm away, while generating laser-induced shockwaves, in most cases, some of the biofilm is disrupted and detached immediately. Generally, the rest of the biofilm detaches after exposure to a number of pulses, i.e. about 10 to about 20 shockwaves. Following the clearing of the biofilm from its host surface, the attached and previously protected bacteria can be seen floating in the liquid medium. The applied shockwave treatment clearly disrupts the biofilms and exposes the protected microorganisms. The exposed biofilm bacteria are accordingly rendered more susceptible to antibiotics or other anti-infective therapeutic modalities.

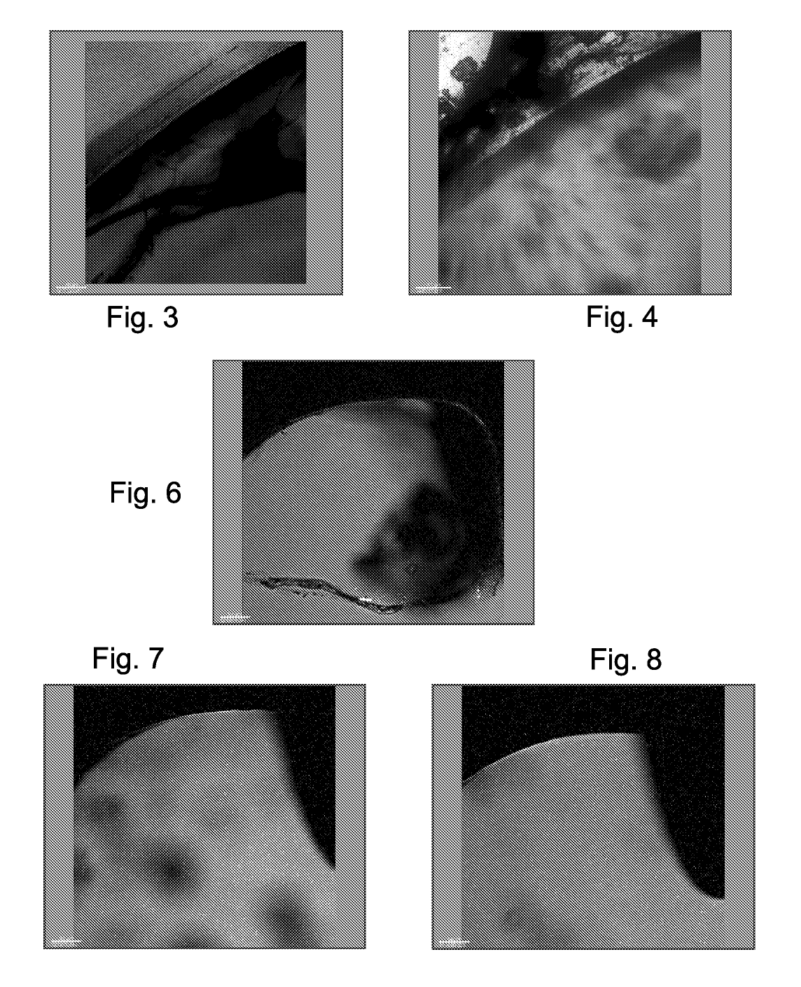

Some examples of images obtainable by the herein described example of biofilm disruption in vitro are shown in FIGS. 3 to 15 of the accompanying drawings. Referring to FIG. 3, the biofilm can be seen as a dark irregular mass in the middle of the image adhering to the geometric outline of a portion of the culture plate in the top of FIG. 3. In FIG. 4, the biofilm can be seen in the foreground to be breaking up and breaking away from the culture plate, as a result of the shockwave treatment. The outline of the culture plate is visible in the background.

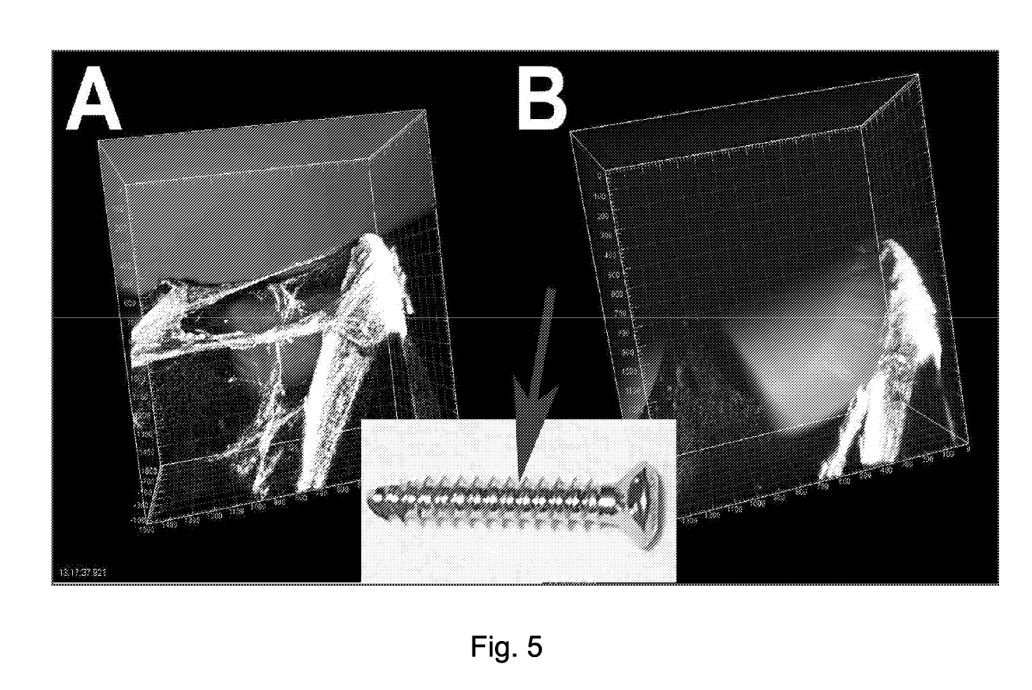

Referring to FIG. 5, cobweb-like masses of light-colored biofilm can be seen in view A, draped across two adjacent threads of the screw. In view B the biofilm on the lefthand thread, and between the threads, has largely been disrupted and dispersed by the shockwave treatment. It is believed that the remaining biofilm attached to the righthand thread could be disrupted and dispersed by further shockwave treatment.

In FIG. 6, the biofilm can be seen as a black irregular mass attached to the righthand side of the light-colored screw thread. A string-like tendril of biofilm is floating in the foreground. In FIG. 7, during shockwave treatment, the biofilm mass can be seen to have broken up into small blobs and in FIG. 8, after the shockwave treatment, the biofilm has almost entirely disappeared.

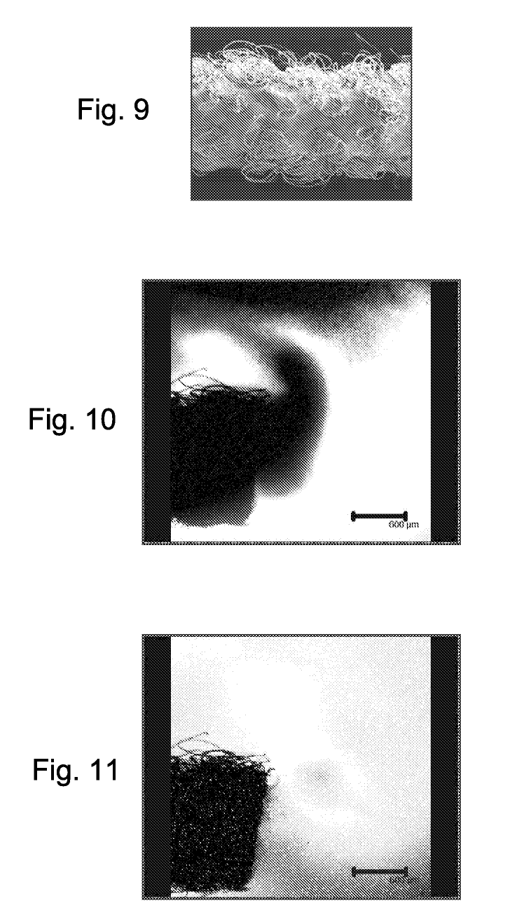

An end-view of the suture fiber shown in FIG. 9 appears on the lefthand side of FIG. 10 as a generally rectangular mass from which some loops and strands of the suture filaments project. A comma-shaped dark mass of biofilm can be seen attached to the righthand side of the suture fiber. The biofilm can be seen to be almost entirely gone from FIG. 11 as a result of the shockwave treatment. The biofilm has been disrupted and the bacteria the biofilm harbored have been removed from the biofilm. An enlarged view of the surface of the suture fiber (not shown) indicates that the suture filaments and the structure of the suture are intact after the shockwave treatment. These fine structures appear visibly undamaged by the powerful shockwaves that substantially eliminated the biofilm.

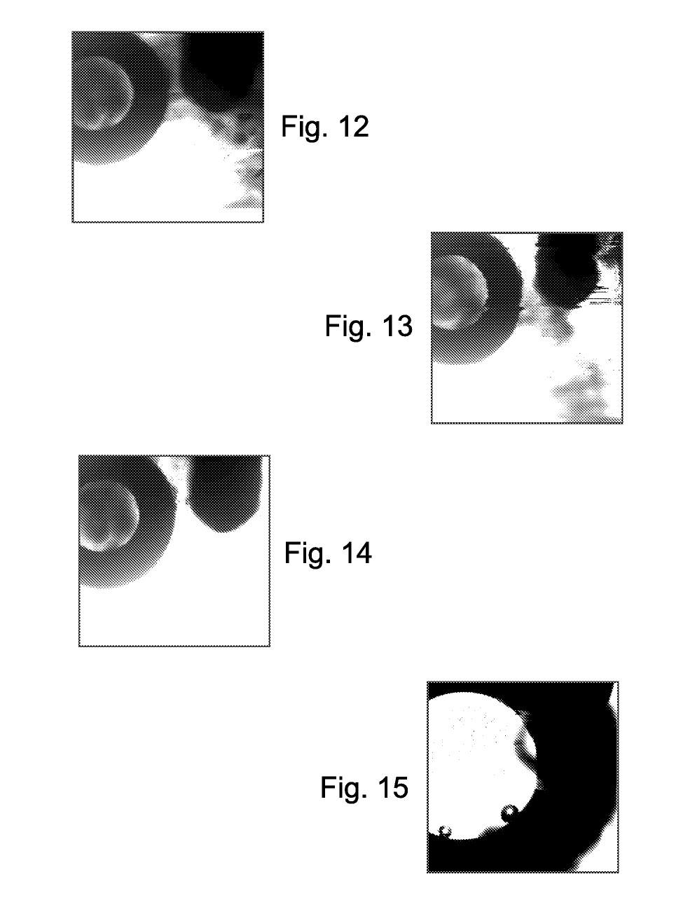

Referring to FIG. 12, biofilm material can be seen inside, outside and around the tympanostomy tube shown on the lefthand side of the image. The dark structure on the righthand side of the tympanostomy tube is the distal tip of the handpiece. In FIG. 13, during shockwave treatment, the biofilm can be seen to be breaking up and dispersing and in FIG. 14, little biofilm remains on the outside of the tympanostomy tube and the adjacent structure. After further shockwave treatment, the biofilm can be seen in FIG. 15 to have been largely removed from the interior of the tympanostomy tube.

No visible damage to the biofilm-infested surgical devices resulting from the shockwave treatments is apparent in these images.

Similar experiments can be performed with other surgical or implantable devices, for example polyurethane foam, a Foley catheter, coated and uncoated nitinol stents, a stainless steel carotid stent and a Penrose drain. Comparable results can be obtained.

The ability to clean and remove biofilm from complex, delicate implant materials, without damage, which can be provided by embodiments of the inventive processes and instruments has useful application in a variety of fields including, for example, for cleaning biofilm-contaminated cardiac implants and associated devices and materials.

As has been referenced herein, the invention includes embodiments wherein the described laser-induced shockwave technology is coupled with endoscopic techniques to facilitate the visualization of, and access to, in vivo biofilms, facilitating the treatment of deeper tissue infections.

Another embodiment of the invention comprises a process for treating biofilms comprising employment of a laser-induced shockwave generating instrument for cellular level ablation or “shaving” of a biofilm resident in vivo. For example, the process can comprise selectively removing a first layer of biofilm with an initial shockwave application, followed by one or more additional shockwave applications to remove additional layers of the biofilm. Each shockwave application can comprise traversing the shockwave across the biofilm by suitably manipulating the instrument. The biofilm can comprise invasive pathogens and the initial shockwave application can expose the invasive pathogens or other microorganisms for destruction by additional shockwaves, or in other desired manner. Subsequent shockwave applications can similarly expose layers of microorganisms deeper in the biofilm. If desired, any suitable antimicrobial therapy can be employed for treating the bacteria or other microorganisms exposed and dispersed after disruption of the biofilm.

A further embodiment of treatment instrument according to the invention comprises illumination means or an illumination device to illuminate the target area to facilitate monitoring of the treatment. If desired, the illumination means can comprise an illumination fiber having a proximal light input end communicating with a light source and having a distal light output end locatable in the vicinity of the treatment site to illuminate the treatment site. The illumination fiber can be movable with the treatment instrument. For example it may be a component of the treatment instrument or it can be a separate device. Illumination means not only can be usefully employed to illuminate concealed treatment sites but may also be useful for treatment of biofilms resident at exposed treatment sites.

Other shockwave or pressure pulse generators that can be employed in the practice of the present invention include piezoelectric, for example piezoceramic, devices, spark discharge devices, electromagnetically or inductively driven membrane pressure shockwave generators or pressure pulse generators and generators that employ pressure currents or jets associated with the transport of material. The pressure pulse generator can be disposed in the treatment instrument or externally in a separate unit connected to the treatment instrument by a transmission line, if desired.

Such other pressure pulse generators may provide useful shockwaves or pressure pulses for biofilm disruption or attenuation, without use of laser or other photic energy, as will be understood by those skilled in the art.

The energy output of some of the herein described embodiments of treatment instrument are flexibly controllable and accurate and well suited to treatment of mammalian host resident biofilms. For example, a number of the parameters of such treatment instruments can be manipulated and varied, including for example, the laser energy and pulse frequency, the optical fiber thickness, the fiber-to-target distance and the geometry of the distal output opening through which the shockwave generates to impinge on a target organ, or other output structure, to vary the output. Any one or more of these and other parameters is, or are, available for adjustment to adapt the applied energy, the energy concentration at the treatment site, the energy duration, the pattern of application and other factors, for any particular treatment. Thus, the invention can provide a user with a flexible treatment process and instrument which can be adapted, without difficulty, to treat biofilms in a variety of locations in a mammalian body.

The processes and instruments of the invention employing laser-generated or other shockwave or pressure wave technology can be useful for disruption or other treatment of host-resident biofilms in otolaryngology and other fields. Some embodiments of the invention are contemplated as having safety parameters when employed for biofilm treatment that allow treatments to be effected in close proximity to sensitive and critical anatomical structures, including for example, cranial nerves and large blood vessels. Furthermore, the mechanical nature of the laser generated shockwave that is applied to the biofilm, in some embodiments of the invention avoids the issues of toxicity and acquired resistance commonly associated with high and/or repeated doses of antibiotics.

The foregoing detailed description is to be read in light of and in combination with the preceding background and invention summary descriptions wherein partial or complete information regarding the best mode of practicing the invention, or regarding modifications, alternatives or useful embodiments of the invention may also be set forth or suggested, as will be apparent to one skilled in the art. Should there appear to be conflict between the meaning of a term as used in the written description of the invention in this specification and the usage in material incorporated by reference from another document, the meaning as used herein is intended to prevail.

Throughout the description, where processes are described as having, including, or comprising specific process steps, it is contemplated that compositions of the present invention can also consist essentially of, or consist of, the recited components, and that the processes of the present invention can also consist essentially of, or consist of, the recited processing steps. It should be understood that the order of steps or order for performing certain actions is immaterial so long as the invention remains operable. Moreover, two or more steps or actions may be conducted simultaneously.

While illustrative embodiments of the invention have been described above, it is, of course, understood that many and various modifications will be apparent to those of ordinary skill in the relevant art, or may become apparent as the art develops, in the light of the foregoing description. Such modifications are contemplated as being within the spirit and scope of the invention or inventions disclosed in this specification.

1. A mammalian biofilm treatment process comprising applying shockwaves to an undesired biofilm present at a treatment site in or on a mammalian host to control the biofilm.

2. A process according to claim 1 wherein controlling the biofilm comprises reducing the mass of, disrupting, attenuating or destroying the biofilm, the biofilm comprising matter foreign to the mammalian host.

3. A process according to claim 1 wherein applying the shockwaves to the biofilm comprises causing one or more pieces of the biofilm to tear away from the residual biofilm or from the treatment site, the applying of the shockwaves optionally comprising oscillating the biofilm.

4. A process according to claim 1 wherein applying the shockwaves comprises impinging a laser beam on to an ionizable target to generate mechanical shockwaves and, optionally, pulsing the laser beam.

5. A process according to claim 1 wherein applying the shockwaves comprises impinging a pulsed laser beam on to an ionizable target to form a plasma adjacent the metallic target and to generate mechanical shockwaves emanating from the plasma and moving away from the ionizable target.

6. A process according to claim 5 wherein applying the shockwaves comprises generating the shockwaves as non-convergent shockwaves and directing the non-convergent shockwaves on to the biofilm resident at the treatment site.

7. A process according to claim 6 comprising employing a treatment instrument to apply the shockwaves, the treatment instrument having a distal tip, wherein the distal tip comprises the metallic target and the plasma is formed at the distal tip, the process further comprising inserting the distal tip of the treatment instrument into the mammalian body and applying the shockwaves while the distal tip is inserted into the mammalian body.

8. A process according to claim 7 comprising manipulating the treatment instrument, optionally by hand, and directing the shockwaves on to the biofilm resident at the treatment site.

9. A process according to claim 8 wherein employing a treatment instrument to apply the shockwaves comprises translating the treatment instrument across the biofilm to incrementally remove the biofilm, the treatment instrument optionally being translated across the biofilm in multiple passes.

10. A process according to claim 8 wherein the treatment instrument comprises an inspection fiber and the process includes inserting the inspection fiber into the mammalian body and monitoring the application of shockwaves by viewing the treatment site via the inspection fiber and manipulating the treatment instrument accordingly.

11. A process according to claim 10 comprising inserting the distal tip into a bodily cavity or introducing the treatment instrument subcutaneously.

12. A process according to claim 10 comprising employing a flexible treatment instrument and a catheter or trocar and inserting the treatment instrument into the vascular system using the catheter or trocar.

13. A process according to claim 10 wherein the treatment instrument comprises a distal port and the process comprises applying the shockwaves through the distal port.

14. A process according to claim 13 comprising manipulating the treatment instrument to position the distal port at a distance from the biofilm at the treatment site in the range of from about 0.5 mm to about 10 mm and effecting the applying of shockwaves with the distal port at said distance from the biofilm.

15. A process according to claim 1 wherein the treatment site is a non-ophthalmologic site and the process comprises controlling the biofilm non-thermolytically or by avoiding delivery of heat to the treatment site or without applying stain to the biofilm or according to a combination of two or all of the foregoing parameters and wherein, optionally controlling the biofilm comprises ablating or disintegrating the biofilm.

16. A process according to claim 4 comprising employing an optical fiber end to output the laser beam and irrigating the optical fiber end with aqueous fluid.

17. A process according to claim 1 wherein the biofilm comprises one or more microorganisms selected from the group consisting of bacteria, fungi, protozoa, archaea and algae and, optionally, is secured to the treatment site by biofilm exopolysaccharide material.

18. A process according to claim 1 wherein the treatment site comprises one or more treatment sites selected from the group consisting of otolaryngological sites; nasal, sinus, and middle ear cavities; pharyngal, tonsillar, dental and periodontal sites; toenails, fingernails; implant sites; cardiac implant sites, endovascular implant sites, orthopedic implant sites, gynecological implant sites, intrauterine device sites, urologic implant sites and urinary catheter sites and the biofilm is adhered to a treatment site.

19. A process according to claim 3 wherein the treatment site comprises one or more treatment sites selected from the group consisting of otolaryngological sites; nasal, sinus, and middle ear cavities; pharyngal, tonsillar, dental and periodontal sites; toenails, fingernails; implant sites; cardiac implant sites, endovascular implant sites, orthopedic implant sites, gynecological implant sites, intrauterine device sites, urologic implant sites and urinary catheter sites; wherein the biofilm is secured to the treatment site by biofilm exopolysaccharide material; and the biofilm comprises one or more microorganisms selected from the group consisting of bacteria, fungi, protozoa, archaea and algae.

20. A process according to claim 1 comprising controlling the application of shockwaves to maintain host tissue at the treatment site intact or free of symptoms of tissue damage or both intact and free of symptoms of tissue damage.

21. A process according to claim 1 comprising employing a treatment instrument to apply the shockwaves and employing aspiration to locate the treatment instrument relatively to the biofilm at the treatment site.

22. A process according to claim 1 wherein applying shockwaves comprises generating shockwaves by employing one or more of a piezoelectric device, a piezoceramic device, a spark discharge device, an electromagnetically driven membrane, an inductively driven membrane, a pressure shockwave generators and a material transport device employing a pressure current or a pressure jet, and optionally, pulsing the shockwaves.

23. A process according to claim 4 wherein applying shockwaves comprises controlling the application of shockwaves to the biofilm by selection of one or more control parameters selected from the group consisting of laser energy pulse width, pulse repetition rate, pulse energy and total energy delivered to the target site, the distance of the output port from the target site and the fiber-to-target distance.

24. A process according to claim 4 wherein applying shockwaves comprises pulsing laser energy impinged on the target to have one or more pulse characteristics selected from the group consisting of a pulse width in the range of from about 2 ns to about 20 ns, a pulse rate of from about 0.5 Hz to about 200 Hz, a pulse energy in a range of from about 2 mJ to about 15 ml of energy per pulse, and a fiber-to-target distance in the range of from about 0.7 to about 1.5 mm.

25. A process according to claim 4 wherein applying shockwaves comprises pulsing laser energy impinged on the target to have a pulse width in the range of from about 2 ns to about 20 ns, a pulse rate of from about 0.5 Hz to about 200 Hz, a pulse energy in a range of from about 2 ml to about 15 ml of energy per pulse and a fiber-to-target distance in the range of from about 0.7 to about 1.5 mm.

26. A process according to claim 4 wherein applying shockwaves comprises pulsing laser energy impinged on the target to have a pulse width of from about 8 to about 12 nanoseconds, a pulse rate of from about 2 Hz to about 6 Hz, and an energy per pulse of from about 6 ml to about 12 ml.

27. A process according to claim 1 wherein the process is accompanied by or followed by local or systemic administration of an antibiotic to control possible infection associated with dispersal of the treated biofilm.

28. A process according to claim 1 wherein applying the shockwaves comprises ablating the biofilm at the cellular level and optionally comprises selectively removing a first layer of biofilm in an initial pass and subsequently removing further layers of biofilm in subsequent passes.

29. A treatment instrument for controlling an undesired biofilm resident at a treatment site in or on a mammalian host, wherein the treatment instrument is adapted to apply shockwaves to the treatment site to control the biofilm.

30. A treatment instrument according to claim 29 comprising an ionizable target for transducing laser energy into shockwaves and an optical fiber extending along the treatment instrument and having a distal end positioned adjacent the ionizable target, the optical fiber being connectable with a pulsed laser energy source to receive pulses of laser energy from the laser energy source and discharge the pulses of laser energy from the distal end of the optical fiber to impinge on the ionizable target, outputting shockwaves.

31. A treatment instrument according to claim 30 configured for outputting shockwaves in a shockwave pattern extending forwardly and distally of the treatment instrument to facilitate directing the shockwaves toward the treatment site.

32. A treatment instrument according to claim 31 disposed in a bodily cavity of the mammalian host or housed by a catheter and disposed subcutaneously in the mammalian host, the treatment instrument having a shockwave output location disposed adjacent the biofilm.

33. A treatment instrument according to claim 32 impinging pulsed laser energy on the biofilm, the pulsed laser energy having one or more pulse characteristics selected from the group consisting of a pulse width in the range of from about 2 ns to about 20 ns, a pulse rate of from about 0.5 Hz to about 200 Hz, a pulse energy in a range of from about 2 mJ to about 15 mJ of energy per pulse.

34. A treatment instrument according to claim 29 and an endoscope for viewing the treatment site the treatment instrument and endoscope being configured for applying shockwaves to the treatment site and for the applying of shockwaves to be modified in response to a view of the treatment site wherein, optionally, the treatment instrument and endoscope are configured for insertion into the mammalian host for treatment of biofilms at non-ophthalmologic sites.
